(19) United States
(12) Patent Application Publication
(10) Pub. No.: US 2024/0099966 A1
(43) Pub. Date: Mar. 28, 2024

(54) EDIBLE PRODUCTS AND COMPOSITIONS FOR EDIBLE PRODUCTS

(71) Applicant: Jelly Drops Limited, London (GB)

(72) Inventors: Claudia ARNOLD, London (GB); Nicholas Dorset HOOTON, London (GB); Lewis Michael HORNBY, London (GB); Dayna Paige JEFFERS, London (GB); Michael FRETWELL, Yunnan (CN)

(21) Appl. No.: 18/553,344

(22) PCT Filed: Mar. 29, 2022
(86) PCT No.: PCT/GB2022/050780

§ 371 Date: Sep. 29, 2023

(30) Foreign Application Priority Data
Mar. 29, 2021 (GB) 2104458.1

(51) Int. Cl. A61K 9/00; A23L 29/238; A23L 29/256; A23L 29/269; A61K 47/26

(52) U.S. Cl. CPC A61K 9/0056; A23L 29/238; A23L 29/256; A23L 29/272; A61K 47/26

(57) ABSTRACT

The present invention relates to edible products and compositions for preparing edible products, particularly edible products suitable for use as confectionery and having therapeutic uses. In particular, it relates to products of the type referred to as gummies and jellies and to compositions and methods for the preparation thereof; and to their use as confectionery and in therapy, in particular in hydration therapies. We describe a gelling composition for preparing a gelled product. The gelling composition comprises or consists of Component A and Component B in a weight ratio of from 1:4 to 1.4:1. Component A comprises at least one rigid gel-forming agent and Component B comprises at least one flexibility modifier. In some examples, Component A and Component B are present in a weight ratio of from 1:4 to 1.5:1, or 1:3 to 1.5:1, or 3:4 to 4:3, or 4:5 to 5:4 or about 1:1.

The present invention relates to edible products and compositions for preparing edible products. In particular, the present invention relates to edible products suitable for use as confectionery and having therapeutic uses. In particular, it relates to products of the type referred to as gummies and jellies and to compositions and methods for the preparation thereof; and to their use as confectionery and in therapy, in particular in hydration therapies.

Dementia is the leading cause of death in the UK and the number of people globally with dementia is set to treble to at least 150 million by 2050.

It is very easy for people with dementia to become dehydrated and their dehydration can have a number of causes or triggers. Many no longer have an awareness of thirst, do not equate drinking with quenching thirst, forget what a cup is for (memory), do not recognise cups for what they are (agnosia), lack the muscular dexterity to use cups (coordination), lack the “muscle memory” to use cups (procedural memory), or lack the ability to plan to use a cup (executive function).

Sources of hydration in liquid format can be difficult for people with dementia to engage with. They are often spilt and can easily be inhaled, causing choking. It can also be very time consuming for carers to keep someone with dementia sufficiently hydrated. Care homes often struggle to fulfil this requirement. The result can be an accelerated decline in residents' conditions that reduces quality of life and exacerbates other care requirements, in turn increasing hospital admissions and strain on the health service providers, such as the NHS. The cost of dementia to the UK is currently estimated to be £26 billion a year, equivalent to £32,250 per person with dementia per year.

Water is needed in the body for a wide range of uses including regulating temperature (through sweating), maintaining blood pressure and eliminating bodily waste, as well as a host of cell reactions. Even mild dehydration can have an effect on people, causing tiredness, headaches and a lack of concentration.

Urinary tract infections (UTIs) are a type of infection common among older people. If a person with a memory impairment or dementia has a UTI, this can cause sudden and severe confusion referred to as delirium. This often leads to hospital admission to be treated with antibiotics and can lead to kidney damage and blood poisoning, which can be fatal.

For people with dementia, the symptoms of dehydration are often mistakenly attributed to their underlying condition, meaning it can easily go unnoticed until it becomes life-threatening.

Often, in a care home setting, there are only short windows of opportunity where a resident will allow a carer to feed them a drink. This results in relatively large volumes of liquid being administered at once. For elderly people with poor kidney function this often results in most of the fluid passing straight through their system, reducing the hydration delivered and increasing urine output. Hydration is related to the amount of fluid absorbed, not the amount ingested.

Given aging societies worldwide, a solution is needed to improve hydration, whilst simultaneously reducing care requirements. The present invention provides hydrating products that enable people with dementia to hydrate more often and independently. It with this in mind that the present invention has been devised.

A common trait of people with dementia is that they enjoy eating sweets. It has been observed that many become excited and enthusiastic when presented with this food type. The finger food format is more easily engaged with and people with dementia intuitively know how to interact with it.

In our earlier application, WO 2020/128531A1, we describe certain compositions for producing edible products containing a large proportion of water within a solid or semi-solid matrix. Suitably, the matrix is formed by a hydrocolloid or hydrocolloidal mixture.

The edible products we described present a person with dementia with a large volume of water in a solid format. This is easier for the person to pick up and engage with compared with liquid or viscous sources of hydration. This is especially the case for people with limited dexterity and coordination. Additionally, by containing the water within a matrix, the product does not ‘pop’, releasing the water suddenly when chewed. For people with reduced cognitive abilities, this may be unexpected and can lead to choking.

Gummies and jellies are a class of confectionery based on using a hydrocolloid to stabilise a bulk sweetener, acting as a bulking agent for the confectionery, to produce a gel-like product having a moisture content of up to about 10-20 wt. %, depending upon the desired firmness of the confectionery product. Lower water content products have a firmer texture. The hydrocolloids crosslink during the heating step of the manufacturing process to form a network structure within which the molecules of the bulk sweetener become trapped to form a semi-solid or gelled structure.

Generally, the bulk sweetener in a gummy or jelly confection is present in an amount of 70-85 wt. % based on the total weight of the gummy or jelly formulation prior to heating. Typically, the bulk sweetener is a mixture of sucrose and glucose syrup. Glucose syrup typically forms 50-60 wt. % of the bulk sweetener mixture, the balance being sucrose. In view of the calorific content of sucrose and glucose, alternative bulk sweeteners have been used to provide reduced-sugar or sugar-free products. Polyols such as maltitol, sorbitol, mannitol, maltitol, isomalt and allulose have been used or proposed. However, overconsumption of polyols can lead to gastrointestinal symptoms, such as diarrhoea, in some individuals. Additionally, whilst polyols are low in calories, they can be converted to glucose in the liver. Accordingly, the use of polyols as bulking agents is far from ideal.

The most common hydrocolloids used are gelatine, starches and pectin. Other hydrocolloids such as agar, gum Arabic and carrageenan, amongst others, are often used in mixtures with other hydrocolloids to modify the visual and textural characteristics of the confectionery product.

Gelatine is perhaps the most common hydrocolloid used in gummies and jellies and is a protein derived from collagen, primarily bovine or porcine in origin. Gelatine is typically used in an amount of about 5 to 10% by weight based on the total of the ingredients for the formulation. Gelatine is obviously unsuitable for consumers on vegetarian or vegan diets.

Accordingly, in our earlier application, we determined that by using a hydrocolloid mixture of agar and a galactomannan polysaccharide, preferably locust bean gum, we can prepare gummy and jelly products in which the bulking agent is water. Compared with known gummies and jellies, which typically have a final water content of no greater than 20 wt. %, our products have achieved water contents of the order of 90 wt. % or more. Such a surprisingly high water-content makes our products exceptionally useful as hydration products, for use in hydration therapies, as described above.

Furthermore, since we can avoid the requirement for bulk sweeteners, our products can satisfy the regulatory requirements to be defined as being sugar-free and as tooth-friendly.

In its broadest aspect, our earlier invention provided a composition for the preparation of a confectionery product comprising 0.2 wt. % to 3 wt. % of at least one polysaccharide gelling agent, the balance being water.

Despite the success of our earlier work, some consumers felt that the texture was not to their liking, commenting that the texture of the product was crumblier than they would have preferred, broke apart too easily or broke into large lumps. Accordingly, we have sought to improve further on our earlier work.

In its broadest aspect, the present invention provides an edible product prepared by gelling a composition comprising or consisting of water in an amount of at least 90 wt % and a gelling composition, wherein the gelling composition is present in an amount of from 0.7 to 2.5 weight percent based on the amount of water, and wherein the gelling composition comprises Component A and Component B in a weight ratio of from 1:4 to 1.4:1, wherein Component A comprises at least one rigid gel-forming agent and Component B comprises at least one flexibility modifier.

The present invention also provides a gelling composition for preparing a gelled product, the gelling composition comprising or consisting of Component A and Component B in a weight ratio of from 1:4 to 1.4:1, wherein Component A comprises at least one firm or rigid gel-forming agent and Component B comprises at least one flexibility modifier.

By the term flexibility modifier, it is intended to encompass materials which have an effect of varying the flexibility of a gel formed by the at least one firm or rigid gel-forming agent of Component A.

The flexibility modifier may be a soft or flexible gel-forming agent or a thickening agent.

In certain examples, a flexibility modifier is a soft, medium or flexible gel-forming agent selected from sodium alginate, calcium alginate, high methoxyl pectin, low methoxyl pectin, iota carrageenan, locust bean gum (carob gum), guar gum, xanthan gum, tara gum, high acyl gellan gum, konjac, methyl cellulose and hydroxypropyl methyl cellulose.

In preferred examples, the flexibility modifier is a gel-forming agent selected from high methyl pectin, low methoxyl pectin, carrageenan iota, locust bean gum (carob gum), guar gum, xanthan gum, tara gum, high acyl gellan gum, konjac and methyl cellulose.

In some examples, a flexibility modifier is a thickening agent, an agent which does not, itself, do form a gel.

In certain examples, the thickening agent is a thickening agent selected from alginic acid, potassium alginate, ammonium alginate, calcium alginate, propane-1,2-diol alginate, carrageenan lambda, processed eucheuma seaweed, clarified guar gum, partially hydrolysed guar gum, tragacanth, acacia gum (gum arabic), karaya gum, soybean hemicellulose, cassia gum, cellulose, ethyl cellulose, hydroxypropyl cellulose, hydroxypropyl methyl cellulose, ethyl methyl cellulose, carboxy methyl cellulose, crosslinked sodium carboxy methyl cellulose and enzymatically hydrolysed carboxy methyl cellulose.

In other examples, the thickening agent is an agent which is capable of forming a gel but which is present in the composition at a concentration or under other conditions at which the agent does not form a gel.

In certain examples, the thickening agent is selected from sodium alginate, agar, carrageenan kappa, carrageenan iota, locust bean gum (carob gum), guar gum standard, xanthan gum, tara gum, low acyl gellan gum, high acyl gellan gum, konjac, high methoxyl pectins, low methoxyl pectins, methyl cellulose and hydroxypropyl methyl cellulose; provided that the agent is present in a concentration insufficient to form a gel.

In preferred examples, the at least one rigid gel-forming agent of Component A is at least one hydrocolloid.

In preferred examples, the at least one flexibility modifier of Component B is at least one hydrocolloid.

In some examples, the gelling composition comprises Component A and Component B in an weight ratio of from 1:4 to 1.5:1, or 1:3 to 1.5:1, or 3:4 to 4:3, or 4:5 to 5:4 or about 1:1.

In some examples, the at least one rigid gel-forming agent shows a deformation at failure, in a 1 wt % solution in water, of less than or equal to 25%.

Preferably, Component A is at least one gelling agent selected from agar, low acyl gellan gum and kappa carrageenan; optionally two gelling agents selected from agar, low acyl gellan gum and kappa carrageenan.

In preferred examples, Component A is two gelling agents selected from agar, low acyl gellan gum and kappa carrageenan and each gelling agent is present in Component A in a weight ratio of about 1.3:1, about 1.25:1 or about 1:1.

Optionally, Component A is three or more gelling agents in approximately equal proportions by weight.

Preferably, the at least one flexibility modifier has a cohesiveness, in a 1 wt % solution in water, of greater than or equal to 0.2 N.

In preferred examples, Component B is at least one of xanthan gum, locust bean gum (carob gum), gum arabic, guar gum, high acyl gellan gum and iota carrageenan.

Preferably, Component B is two components selected from xanthan gum, locust bean gum (carob gum), gum arabic, guar gum, high acyl gellan gum and iota carrageenan and each gelling agent is present in Component B in a weight ratio of about 2:1 to about 1:1, optionally about 2:1, 1.5:1 or 1:1.

Optionally, Component B is three or more components in approximately equal proportions by weight.

The present invention also provides a composition for preparing a gelled product, the composition comprising water in an amount of at least 90 wt. % and a gelling composition as defined above in an amount of from 0.7 to 2.5 wt. % based on the total weight of the composition.

In certain preferred examples, the composition comprises locust bean gum (also known as carob gum) in an amount of 0.25 to 0.4 wt %.

In certain preferred examples, the composition comprises low acyl gellan gum, optionally in an amount of from 0.1 to 0.5 wt % or 0.2 to 0.4 wt %.

In certain preferred examples, the composition comprises agar, optionally in an amount of from 0.25 to:1.0 wt % or 0.3 to 0.8 wt %.

In certain examples, the composition comprises gum arabic, optionally in an amount of from 0.1 to:0.5 wt % or from 0.2 to 0.4 wt %.

In certain preferred examples, the composition further comprises xanthan gum, optionally in an amount of from 0.1 to:0.5 wt % or 0.2 to 0.4 wt %. In certain examples, xanthan gum is combined with a galactomannan. Optionally, the galactomannan is selected from guar gum, locust bean gum, tara gum and cassia gum.

In certain examples, the composition comprises: i) agar and carrageenan; preferably agar in an amount of about 0.4-0.5 wt % and a mixture of iota and kappa carrageenan in approximately equal proportions by weight in a combined amount of about 0.7-0.8 wt %; ii) gellan gum and xanthan gum, preferably low acyl gellan gum in an amount of 0.2-0.4 wt % and xanthan gum in an amount of 0.2-0.4 wt %; iii) agar and xanthan gum, preferably agar in an amount of 0.3 to 0.8 wt % and xanthan gum in an amount of 0.3 to 0.4 wt %; or iv) xanthan gum and carrageenan; preferably xanthan gum in an amount of about 0.3-0.5 wt % and a mixture of iota and kappa carrageenan in approximately equal proportions by weight in a combined amount of about 0.7-0.9 wt %;

In certain preferred examples, the composition comprises gellan gum in an amount of 0.3-0.6 wt %, gum arabic in an amount of 0.1-0.5 wt % and xanthan gum in an amount of 0.3-0.5 wt %; and/or agar, optionally agar in an amount of 0.25 to 0.5 wt %, preferably in an amount of 0.3 to 0.4 wt %. Optionally, the composition comprises: i) gellan gum in an amount of about 0.4 wt %, agar in an amount of about 0.4 wt %, gum arabic in an amount of about 0.2 wt % and xanthan gum in an amount of about 0.4 wt %; ii) gellan gum in an amount of about 0.4 wt %, agar in an amount of about 0.4 wt %, gum arabic in an amount of about 0.4 wt % and xanthan gum in an amount of about 0.4 wt %; iii) gellan gum in an amount of about 0.5 wt %, agar in an amount of about 0.4 wt %, gum arabic in an amount of about 0.4 wt % and xanthan gum in an amount of about 0.4 wt %; or iv) gellan gum in an amount of about 0.4 wt %, agar in an amount of about 0.3 wt %, gum arabic in an amount of about 0.3 wt % and xanthan gum in an amount of about 0.4 wt %.

Preferably the gellan gum is low acyl gellan gum.

In preferred embodiments, the composition further comprises soluble fibre, optionally in an amount of 0.2 to 1.0 wt %, or 0.3 to 0.7 wt %.

In preferred embodiments, the composition further comprises maltodextrin, optionally in an amount of from 0.9 to 2.2 wt %, preferably 1 to 2 wt %.

In preferred embodiments, the composition further comprises at least one electrolyte component in an amount of up to about 3 wt. %. Optionally, the at least one electrolyte component is present in a total electrolyte amount of from 0.2 wt. % to 3 wt. %, or 0.5 wt. % to 3 wt. % or 1 wt. % to 3 wt. %.

Preferably, the at least one electrolyte component comprises sodium, potassium, calcium and/or magnesium ions. Optionally, the at least one electrolyte component is at least one of sodium chloride, potassium chloride disodium hydrogen citrate, potassium sulphate, calcium hydrogen phosphate, magnesium oxide, calcium carbonate, tricalcium phosphate and magnesium carbonate.

Optionally, the water of the composition is hard water and the at least one electrolyte component is a component of the hard water.

Optionally, the at least one electrolyte component is at least one of sodium chloride, potassium chloride disodium hydrogen citrate, potassium sulphate, calcium hydrogen phosphate, magnesium oxide, calcium carbonate, tricalcium phosphate and magnesium carbonate.

Preferably, the composition has a pH in the range of about 3 to 7.

In preferred embodiments, the composition further comprises lactic acid, optionally in an amount of from 0.5 to 1.0 wt %.

In preferred embodiments, the composition further comprises an acid buffer composition, optionally in an amount of from 0.5 to 0.8 wt %.

In preferred embodiments, the composition further comprises at least one non-nutritive sweetener; optionally wherein the non-nutritive sweetener is present in an amount of up to about 0.1 wt. %, preferably up to about 0.05 wt. %, more preferably about 0.025 wt. %.

In preferred embodiments, the composition further comprises i) one or more nutrient compositions, optionally one or more vitamins, minerals, fats or amino acids; and/or ii) at least one acidulant, optionally at least one of malic acid and citric acid, further optionally in an amount of up to about 1.0 wt. %.

In certain embodiments, the composition further comprises at least one flavourant, preferably in an amount of up to about 0.5 wt. %, more preferably up to about 0.4 wt. %; and/or at least one colourant, preferably in an amount of up to about 0.5 wt. %, more preferably up to about 0.2 wt. %, more preferably up to about 0.15 wt. %.

In preferred embodiments, the water content is at least 92 wt. %, preferably at least 94 wt. %.

The above and other aspects of the present invention will now be described in further detail, by way of example only, with reference to the accompanying examples.

In the present disclosure, except where the context makes clear, the terms firm, hard, rigid and brittle are considered to be synonymous when describing the gel texture of Component A. Equally, the terms soft, elastic and flexible are considered to be synonymous in the context of the gel texture of Component B. The term medium will be understood accordingly.

Characterisation of the properties of gels and the components from which they are formed are well known in the art. The reader is referred, for example, to the Handbook of Hydrocolloids, Second Edition, edited by G. O. Phillips and P. A. Williams; and to Food Polysaccharides and Their Applications, Second Edition, edited by Alistair M. Stephen, Glyn O. Phillips and Peter A. Williams. Reference is made particularly to FIG. 1.10 and the accompanying commentary.

Except where the context indicates, all percentages are given as percentages by weight of the total composition and, apart from references to water or ingredients which are inherently liquid at the relevant processing temperature, references are to percentages by weight of the dry ingredients as percentages of the total composition.

The ingredients are all food-grade ingredients.

The components of Component A defined above are chosen primarily for their characteristics for imparting stiffness to the final product. The preferred components show a stiffness, as defined by deformation at failure of a 1 wt % solution in water of about 25% or lower (Nussinovitch, A., Kaletunc, G., Normand, M. D. and Peleg, M. (1990), Recoverable Work Versus Asymptotic Relaxation Modulus In Agar, Carrageenan And Gellan Gels, Journal of Texture Studies, 21: 427-438. https://doi.org/10.1111/j.1745-4603.1990.tb00492.x).

By the term flexibility modifier, it is intended to encompass materials which have an effect of varying the flexibility of a gelled composition formed by Component A alone.

The components of Component B defined above are chosen to provide a cohesiveness to the gelled compositions to provide a pleasant mouthfeel to the final product. The preferred components show a cohesiveness, in a 1 wt % solution in water, of at least about 0.2 N (Chiara Cevoli, Federica Balestra, Luigi Ragni, Angelo Fabbri, Rheological characterisation of selected food hydrocolloids by traditional and simplified techniques, Food Hydrocolloids, 33(1) 2013, pp 142-150, https://doi.org/10.1016/j.foodhyd.2013.02.022.)

### Texture Analysis

Moulded jellies were subjected to texture analysis to analyse its textural parameters of firmness, springiness and bite force. Samples were analysed both prior to pasteurisation and post-pasteurisation.

A Stable Micro System TA XT Plus texture analyser fitted with a 5 kg load cell was used in compression mode. Fifteen individual pieces were measured for each sample.

The Full Compression test compressed the sample using a 25 mm diameter cylinder until the sample broke apart. The settings used were:

| | |
|---|---|
| Pre-test speed | 1 mm/s |
| Test speed | 1 mm/s |
| Post-test speed | 10 mm/s |
| Strain | 60% |
| Trigger force | 5 g |

Firmness is defined, in this test, as the maximum peak force achieved in the test and Fracturability as the distance of this peak. Elasticity is calculated as the gradient of the trace between 5 mm and the Peak force. The force was also determined at 3 mm.

### Texture Profile Analysis (TPA)

The Texture Profile Analysis is a double compression test used to calculate the springiness of a material. The texture profile is an important consideration for both our confectionery products and our hydration therapy products. For example, if the product is too flexible or frangible, it can be difficult to pick up, especially for people with limited dexterity. Accordingly, satisfactory firmness, factorability and elasticity are important considerations. Additionally, the product must be easy to chew and able to break apart and rupture easily in the mouth, with very little force or chewing, especially for people with weak jaw muscles and for those who may have lost some or all of their teeth. Yet, the products need to be able to withstand the production and packaging processes.

Texture profile analysis of gelled systems is well known in the art. Reference is made, for example, to the paper by Pons and Fiszman—Instrumental texture profile analysis with particular reference to gelled systems—Journal of Texture Studies 27 (1996) 597-624.

The samples were compressed using a 45 mm flat disk probe to 30% of its height (the strain). The settings used are shown below

| | |
|---|---|
| Pre-test speed | 5 mm/s |
| Test speed | 5 mm/s |
| Post-test speed | 5 mm/s |
| Strain | 30% |
| Trigger force | 5 g |

Springiness is defined, in this test, as how much the sample springs back after the first compression (expressed a percentage of its height).

### Bite Test

This test replicates the first bite into the sample by cutting the sample with a ‘tooth probe’. The Bite force is defined, in this case, as the maximum force required to cut through the sample.

The settings used are shown below.

| | |
|---|---|
| Pre-test speed | 1 mm/s |
| Test speed | 1 mm/s |
| Post-test speed | 10 mm/s |
| Strain | 60% |
| Trigger force | 5 g |

### REFERENCE EXAMPLE 1 (PREPARATIVE EXAMPLE 1 OF WO 2020/128531)

To make a 1000 g batch.

### Ingredients:

Hydrocolloid mixture

| | | |
|---|---|---|
| Agar (Gelidium) | 13 | g |
| Locust Bean Gum | 3.9 | g |
| Maltodextrin | 20 | g |
| Sucralose | 0.25 | g (to taste) |

Electrolyte mixture

| | | |
|---|---|---|
| Sodium chloride | 0.84 | g |
| Potassium chloride | 0.72 | g |
| Potassium sorbate | 1 | g |
| Sodium benzoate | 0.88 | g |

Acidic buffer mixture

| | | |
|---|---|---|
| Trisodium citrate | 1.4 | g |
| Malic acid | 3.8 | g (to taste) |
| Citric acid | 3.8 | g (to taste) |
| Flavouring | Up to 4 | g (as required) |
| Colouring | Up to1.5 | g (as required) |
| Water | Balance | |

The gelling components, agar and locust bean gum powders, were mixed to form a hydrocolloid mixture. A salt mixture was prepared containing the sodium chloride, potassium chloride, sodium benzoate and potassium sorbate. An acidic buffer mixture was prepared by mixing the trisodium citrate, malic acid and citric acid.

The hydrocolloid mixture was added, together with sucralose and maltodextrin, with sieving to 944.91 g of water. The mixture was heated slowly, with stirring, to the activation temperature of the agar (100° C.). The mixture was boiled at this temperature for five minutes, with stirring, to activate the agar and locust bean gum.

The mixture was allowed to cool to around 80° C. (over a period of about 20 minutes). The salt mixture was added as a solution to the activated solution with thorough mixing.

The temperature of the solution was allowed to cool to about 70° C. and the acidic buffer mixture was added, with stirring. When agar is exposed to hot and acidic conditions, its gelling properties diminish. The acid buffer mixture counteracts this effect.

The colourings and flavourings are added to the mixture in the appropriate quantities to achieve the desired taste and colour; and at a temperature appropriate to the flavour and colour ingredients. Conveniently, powdered colourings and flavourings can be diluted with a small volume of water prior to addition, to improve uniform dispersion and reduce losses.

The mass of the mixture was weighed and water added as necessary to maintain the correct water percentage and total mass of the formulation.

Whilst the mixture remained at a temperature above the setting temperature of the agar (about 40° C. for Gelidium agar), the mixture was poured into moulds of the desired shape and the moulds were refrigerated until the moulded jellies were sufficiently stable to retain their moulded shape, typically after about 10 minutes at 4° C. for a moulded jelly, moulded as a teardrop shape, having a volume of about 13 ml and having a length of about 45 mm and a diameter at its maximum point of about 30 mm.

Half of the samples were subjected to a pasteurising step as described in WO 2020/128531.

### INVENTIVE EXAMPLE 1

To make a 1 kg batch.

### Ingredients:

| Gelling mixture | | |
|---|---|---|
| Agar (Gelidium) | 3.2 | g |
| Gellan (low acyl) | 4 | g |
| Locust Bean Gum | 3.2 | g |
| Gum Arabic | 3.2 | g |
| Xanthan gum | 3.2 | g |
| Soluble Fibre | 3.2 | g |
| Maltodextrin | 10.4 | g |
| Sucralose | 0.28 | g (to taste) |
| Electrolyte mixture | 25 | g |
| Acidic buffer mixture | 6.26 | g |
| Lactic acid | 6.26 | g |
| Water | 931.8 | g |

The soluble fibre was Nutriose (trade mark) soluble fibre, obtainable from Roquette Freres. The gum arabic, and xanthan gum were obtained from Special Ingredients Limited. The electrolyte mixture and acidic buffer mixture were as for Reference Example 1 above.

The gelling components were combined with the Sucralose and added to approximately 300 ml of hot water with thorough stirring. Approximately 150 ml boiling water was added and the mixture heated, with stirring, to at least 97° C. Approximately 200 ml of cold water was added. Once the temperature of the mixture was less than about 80° C., the electrolyte mixture was added, with stirring, which continued for three minutes. The acid buffer and the lactic acid were both prepared as solutions and added to the mixture. A further approximately 125 ml warm water was added to the mixture, with stirring, which continued for three minutes. Further warm water was added to take the total mass of the mixture to 1 kg.

The mixture was decanted into moulds as described for Reference Example 1.

Once set, the moulded products of Reference Example 1 and Inventive Example 1 were subjected to texture analysis tests as discussed above. The results are given in Table 1 below.

TABLE 1
Texture Analysis

| | Reference | | Inventive |
|---|---|---|---|
| | Example 1 | | Example 1 |
| | Non- | | Non- |
| | pasteurised | Pasteurised | pasteurised |
| Test | | | |
| Firmness/g | 955 | 1502 | 1280 |
| Fracturability/mm | 7.8 | 8.0 | 10 |
| Elasticity/g/mm | 209 | 329 | 206 |
| Bite Force/g (force) | 148 | 225 | 150 |
| Springiness | 93 | 96 | 96 |
| 3 mm Force | 120 | 175 | 90 |

As can be seen from these results, the innovative gelling mixture provides a significant improvement to firmness over the unpasteurised examples of Reference Example 1, whilst retaining similar levels of elasticity and bite force. Furthermore, Inventive Example 1 provides an increase in fracturability over both the unpasteurised and pasteurised examples of Reference Example 1, thus showing considerably lower crumbling in the finished product than was shown by the Reference Example of WO 2020/128531.

### COMPARATIVE EXAMPLES

To confirm that the advantageous texture properties were the direct consequence of the rigid gel-forming agents and flexibility modifiers, Inventive Example 1 was repeated—i) by omitting the maltodextrin; ii) by omitting the soluble fibre; and iii) omitting the electrolyte, lactic acid and buffer. The examples confirmed that neither the maltodextrin, nor the fibre, nor the electrolyte, lactic acid and buffer combination, had any impact on the texture of the drops.

Similar results were shown with the following further inventive examples, prepared in accordance with the method of Inventive Example 1. All values are given as percentages by weight and italicized components represent gelling agents.

### INVENTIVE EXAMPLE 2

### Ingredients

| | |
|---|---|
| Water | 92.41 |
| Agar | 0.45 |
| lota carrageenan | 0.365 |
| Kappa carrageenan | 0.365 |
| Soluble Fibre | 0.68 |
| Locust bean gum | 0.25 |
| Maltodextrin | 1.7 |
| Sucralose | 0.028 |
| Electrolytes | 2.5 |
| Acid Buffer | 0.626 |
| Lactic acid | 0.626 |

### INVENTIVE EXAMPLE 3

### Ingredients

| | |
|---|---|
| Water | 92.82 |
| Gellan (low acyl) (Kelcogel F) | 0.2 |
| Maltodextrin | 2 |
| Soluble Fibre | 0.6 |
| Xanthan | 0.2 |
| Locust bean gum | 0.4 |
| Sucralose | 0.028 |
| Electrolytes | 2.5 |
| Acid Buffer | 0.626 |
| Lactic acid | 0.626 |

### INVENTIVE EXAMPLE 4

### Ingredients

| | |
|---|---|
| Water | 92.42 |
| Agar | 0.8 |
| Maltodextrin | 1.6 |
| Soluble Fibre | 0.6 |
| Xanthan | 0.4 |
| Locust bean gum | 0.4 |
| Sucralose | 0.028 |
| Electrolytes | 2.5 |
| Acid Buffer | 0.626 |
| Lactic acid | 0.626 |

### INVENTIVE EXAMPLE 5

### Ingredients

| | |
|---|---|
| Water | 92.42 |
| Gellan (low acyl) | 0.4 |
| Agar | 0.4 |
| Maltodextrin | 1.6 |
| Soluble Fibre | 0.6 |
| Xanthan | 0.4 |
| Locust bean gum | 0.4 |
| Sucralose | 0.028 |
| Electrolytes | 2.5 |
| Acid Buffer | 0.626 |
| Lactic acid | 0.626 |

### INVENTIVE EXAMPLE 6

### Ingredients

| | |
|---|---|
| Water | 92.42 |
| lota carrageenan | 0.4 |
| Kappa carrageenan | 0.4 |
| Maltodextrin | 1.6 |
| Soluble Fibre | 0.6 |
| Xanthan | 0.4 |
| Locust bean gum | 0.4 |
| Sucralose | 0.028 |
| Electrolytes | 2.5 |
| Acid Buffer | 0.626 |
| Lactic acid | 0.626 |

### INVENTIVE EXAMPLE 7

### Ingredients

| | |
|---|---|
| Water | 92.42 |
| Gellan (low acyl) | 0.4 |
| Agar | 0.4 |
| Maltodextrin | 1.6 |
| Soluble Fibre | 0.4 |
| Gum Arabic | 0.2 |
| Xanthan | 0.4 |
| Locust bean gum | 0.4 |
| Sucralose | 0.028 |
| Electrolytes | 2.5 |
| Acid Buffer | 0.626 |
| Lactic acid | 0.626 |

### INVENTIVE EXAMPLE 8

### Ingredients

| | |
|---|---|
| Water | 92.42 |
| Gellan (low acyl) | 0.4 |
| Agar | 0.4 |
| Maltodextrin | 1.4 |
| Soluble Fibre | 0.4 |
| Gum Arabic | 0.4 |
| Xanthan | 0.4 |
| Locust bean gum | 0.4 |
| Sucralose | 0.028 |
| Electrolytes | 2.5 |
| Acid Buffer | 0.626 |
| Lactic acid | 0.626 |

### INVENTIVE EXAMPLE 9

### Ingredients

| | |
|---|---|
| Water | 92.42 |
| Gellan (low acyl) | 0.5 |
| Agar | 0.4 |
| Maltodextrin | 1.3 |
| Soluble Fibre | 0.4 |
| SI Xanthan | 0.4 |
| Locust bean gum | 0.4 |
| SI Gum Arabic | 0.4 |
| Sucralose | 0.028 |
| Electrolytes | 2.5 |
| Acid Buffer | 0.626 |
| Lactic acid | 0.626 |

As with our previous invention, whilst the present invention has been devised particularly to provide a high water-content product for the purposes of hydration therapies, it will be appreciated from the above discussion that the compositions and methods are equally applicable to the manufacture of confectionery. In the field of confectionery, water contents of the order of 90 wt. % or more may not be required. In other words, it may not be necessary to replace all of the bulk sweetener of a typical confectionery product with water. Accordingly, particularly in the field of gummy and jelly confectionery, water contents of 20 wt. % or more may, for certain products, be chosen. Preferably, the water content of a confectionery product in accordance with the present invention is at least 25 wt. % or more, 40 wt. % or more, 50 wt. % or more, 60 wt. % or more or 70 wt. % or more. Most preferably, a confectionery product in accordance with the present invention comprises at least about 80 wt. % water (or other water-based beverage).

The present invention provides a gummy and jelly formulation in which the conventional bulk sweetener bulking agent is effectively replaced entirely by water as the bulking agent, giving a high water-content confection. The composition provides a sugar-free confection without relying on polyols or other bulk sweeteners having negative gastrointestinal effects. Consequently, the present invention provides gummy and jelly products and methods for the production of gummy and jelly products which avoid the need for a curing or stoving process and which can be subjected to a pasteurisation step, whilst producing a product which is sufficiently firm, even when moulded to a relatively large size, to be held by hand.

The present invention is all the more surprising in that such a product can be produced which has excellent fracturability and other textural properties and a water content of up to about 95-96 wt. % or higher.

Additionally, by adjusting the balance of gelling and thickening agents, we have been able to develop a composition which retains and improves on the structural resilience of the products of our earlier work, in terms of avoiding deformation under its own weight and retaining its firmness for ease of picking up. By balancing gelling agents which provide structural properties with components which provide cohesiveness and springiness to the product, we have been able to reduce the relative amount of structural gelling agent, such as agar, and improve the mouthfeel of the product.

Furthermore, in preferred embodiments, in which the structural properties are formed by a combination of gelling agents, such as agar and gellan gum, we have been able to balance the negative mouthfeel effects of both components. Agar gels are typically brittle and break into small pieces. Low acyl gellan gum gels are also brittle but are typically mushy and grainy and have an unpleasant mouthfeel. However, by combining two gelling agents, we have been able to maintain the necessary structural integrity to the product whilst reducing the negative mouthfeel effects of both components. Furthermore, the combination of agar and gellan together provides an overall stronger product than either component alone. Further improvements have been possible by adjustment of the components providing cohesiveness to the compositions, such as by combining xanthan gum and locust bean gum, providing a smoother and more elastic mouthfeel without making the jelly excessively firm or ‘gummy’ and difficult to chew.

For example, agar as the only brittle gel gives a fractured and jagged bite which, in the mouth, causes it to crumble into cubic pieces. The addition of gellan as a second brittle gel enhances the rigidity of the product as, together, they have been found to withstand a higher stress relative to agar or gellan alone. Gellan reinforces the structure of the drops but cannot be used alone as the resulting texture is then unpleasantly grainy. The combination of the two rigid gel forming materials, agar and gellan together, has a clean cut and would not be described as ‘crumbly’, but may be considered to be slightly grainy to some tastes. This is improved further by the addition of the Component 2 agents to increase springiness and give the product a bite with a smooth, clean cut and no graininess.

Overall, as demonstrated by the fracturability analysis, the bite or cut of products manufactured with the inventive compositions is much cleaner and cohesive.

1. A gelling composition for preparing a gelled product, the gelling composition comprising or consisting of Component A and Component B in a weight ratio of from 1:4 to 1.4:1, wherein Component A comprises at least one firm or rigid gel-forming agent and Component B comprises at least one flexibility modifier.

2. A composition as claimed in claim 1 wherein the gelling composition comprises Component A and Component B in a weight ratio of from 1:4 to 1.5:1, or 1:3 to 1.5:1, or 3:4 to 4:3, or 4:5 to 5:4 or about 1:1.

3. A composition as claimed in claim 1 or claim 2 wherein the at least one rigid gel-forming agent shows a deformation at failure, in a 1 wt. % solution in water, of less than or equal to 25%.

4. A composition as claimed in any preceding claim wherein Component A is at least one gelling agent selected from agar, low acyl gellan gum and kappa carrageenan.

5. A composition as claimed in claim 4 wherein Component A is two gelling agents selected from agar, low acyl gellan gum and kappa carrageenan.

6. A composition as claimed in claim 5 wherein each gelling agent of Component A is present in a weight ratio of about 1.2:1 to 1:1.2, optionally about 1.1:1 to 1:1.1 or about 1 to about 1

7. A composition as claimed in claim 4 wherein Component A comprises agar, low acyl gellan gum and kappa carrageenan in approximately equal proportions by weight.

8. A composition as claimed in any preceding claim wherein the at least one flexibility modifier of Component B has a cohesiveness, in a 1 wt. % solution in water, of greater than or equal to 0.2 N.

9. A composition as claimed in any preceding claim wherein the flexibility modifier is a soft or medium gel-forming agent or a thickening agent.

10. A composition as claimed in claim 9 wherein the flexibility modifier is a soft or medium gel forming agent selected from sodium alginate, calcium alginate, high methoxyl pectin, low methoxyl pectin, iota carrageenan, locust bean gum (carob gum), guar gum, xanthan gum, tara gum, high acyl gellan gum, konjac, methyl cellulose and hydroxypropyl methyl cellulose.

11. A composition as claimed in claim 9 wherein the flexibility modifier is a thickening agent selected from alginic acid, potassium alginate, ammonium alginate, calcium alginate, propane-1,2-diol alginate, carrageenan lambda, processed eucheuma seaweed, clarified guar gum, partially hydrolysed guar gum, tragacanth, acacia gum (gum arabic), karaya gum, soybean hemicellulose, cassia gum, cellulose, ethyl cellulose, hydroxypropyl cellulose, hydroxypropyl methyl cellulose, ethyl methyl cellulose, carboxy methyl cellulose, crosslinked sodium carboxy methyl cellulose, enzymatically hydrolysed carboxy methyl cellulose, sodium alginate, agar, carrageenan kappa, carrageenan iota, locust bean gum (carob gum), guar gum standard, xanthan gum, tara gum, low acyl gellan gum, high acyl gellan gum, konjac, high methoxyl pectins, low methoxyl pectins, methyl cellulose and hydroxypropyl methyl cellulose.

12. A composition as claimed in any preceding claim wherein Component B is at least one agent selected from xanthan gum, locust bean gum (carob gum), gum arabic, high acyl gellan gum and iota carrageenan.

13. A composition as claimed in claim 12 wherein Component B is or comprises two or three agents selected from xanthan gum, locust bean gum (carob gum), gum arabic, high acyl gellan gum, guar gum and iota carrageenan.

14. A composition as claimed in claim 13 wherein Component B is or comprises two agents selected from xanthan gum, locust bean gum (carob gum), gum arabic, high acyl gellan gum, guar gum and iota carrageenan and each gelling agent is present in a weight ratio of about 1.2:1 to 1:1.2.

15. A composition as claimed in claim 14 wherein the two agents are present in a weight ratio of about 1.1:1 to 1:1.1 or about 1 to about 1.

16. A composition as claimed in claim 13 wherein Component B is or comprises three agents in approximately equal proportions by weight.

17. A composition for preparing a gelled product, the composition comprising water in an amount of at least 90 wt. % and a gelling composition as claimed in any preceding claim in an amount of from 0.7 to 2.5 wt. % based on the total weight of the composition.

18. A composition as claimed in claim 17 wherein the composition comprises locust bean gum (carob gum) in an amount of 0.25 to 0.4 wt. %.

19. A composition as claimed in claim 17 or claim 18 comprising low acyl gellan gum.

20. A composition as claimed in claim 19 comprising low acyl gellan gum in an amount of from 0.1 to:0.5 wt. % or 0.2 to 0.4 wt. %.

21. A composition as claimed in any one of claims 17 to 20 comprising agar.

22. A composition as claimed in claim 21 comprising agar in an amount of from 0.25 to:1.0 wt. % or 0.3 to 0.8 wt. %.

23. A composition as claimed in any one of claims 17 to 22 comprising gum arabic, optionally in an amount of from 0.1 to 0.5 wt. % or from 0.2 to 0.4 wt. %.

24. A composition as claimed in any one of claims 17 to 23 comprising xanthan gum.

25. A composition as claimed in claim 24 comprising agar in an amount of from 0.1 to:0.5 wt. % or 0.2 to 0.4 wt. %.

26. A composition as claimed in claim 24 or claim 25 comprising xanthan gum in combination with a galactomannan, optionally guar gum or Tara gum.

27. A composition as claimed in claim 17 comprising agar and carrageenan.

28. A composition as claimed in claim 27 comprising agar in an amount of about 0.4-0.5 wt. % and a mixture of iota and kappa carrageenan in approximately equal proportions by weight in a combined amount of about 0.7-0.8 wt. %.

29. A composition as claimed in claim 17 comprising gellan gum and xanthan gum.

30. A composition as claimed in claim 29 wherein the gellan gum is low acyl gellan gum.

31. A composition as claimed in claim 29 or claim 30 comprising gellan gum in an amount of 0.2-0.4 wt. % and xanthan gum in an amount of 0.2-0.4 wt. %.

32. A composition as claimed in claim 17 comprising agar and xanthan gum.

33. A composition as claimed in claim 32 comprising agar in an amount of 0.3 to 0.8 wt. % and xanthan gum in an amount of 0.3 to 0.4 wt. %.

34. A composition as claimed in claim 17 comprising xanthan gum and carrageenan.

35. A composition as claimed in claim 34 comprising xanthan gum in an amount of about 0.3-0.5 wt. % and a mixture of iota and kappa carrageenan in approximately equal proportions by weight in a combined amount of about 0.7-0.9 wt. %.

36. A composition as claimed in claim 17 comprising gellan gum in an amount of 0.3-0.6 wt. %, gum arabic in an amount of 0.1-0.5 wt. % and xanthan gum in an amount of 0.3-0.5 wt. %.

37. A composition as claimed in claim 38 further comprising agar, optionally in an amount of 0.25 to 0.5 wt. %, or 0.3 to 0.4 wt. %.

38. A composition as claimed in claim 17 comprising gellan gum in an amount of about 0.4 to about 0.5 wt. %, agar in an amount of about 0.3 to about 0.4 wt. %, gum arabic in an amount of about 0.2 to about 0.4 wt. % and xanthan gum in an amount of about 0.4 wt. %.

39. A composition as claimed in claim 38 comprising:
i) gellan gum in an amount of about 0.4 wt. %, agar in an amount of about 0.4 wt. %, gum arabic in an amount of about 0.2 wt. % and xanthan gum in an amount of about 0.4 wt. %;
ii) gellan gum in an amount of about 0.4 wt. %, agar in an amount of about 0.4 wt. %, gum arabic in an amount of about 0.4 wt. % and xanthan gum in an amount of about 0.4 wt. %;
iii) gellan gum in an amount of about 0.5 wt. %, agar in an amount of about 0.4 wt. %, gum arabic in an amount of about 0.4 wt. % and xanthan gum in an amount of about 0.4 wt. %; or
iv) gellan gum in an amount of about 0.4 wt. %, agar in an amount of about 0.3 wt. %, gum arabic in an amount of about 0.3 wt. % and xanthan gum in an amount of about 0.4 wt. %.

40. A composition as claimed in any one of claims 17 to 39 further comprising soluble fibre, optionally in an amount of 0.2 to 1.0 wt. %, or 0.3 to 0.7 wt. %.

41. A composition as claimed in any one of claims 17 to 40 further comprising maltodextrin, optionally in an amount of from 0.9 to 2.2 wt. %, preferably 1 to 2 wt. %.

42. A composition as claimed in any one of claims 17 to 41 further comprising at least one electrolyte component in a total electrolyte amount of up to about 3 wt. %.

43. A composition as claimed in claim 42 comprising at least one electrolyte component in a total electrolyte amount of from 0.2 wt. % to 3 wt. %, or 0.5 wt. % to 3 wt. % or 1 wt. % to 3 wt. %.

44. A composition as claimed in claim 42 or claim 43 wherein the at least one electrolyte component comprises sodium, potassium, calcium and/or magnesium ions.

45. A composition as claimed in claim 44 wherein the at least one electrolyte component is at least one of sodium chloride, potassium chloride disodium hydrogen citrate, potassium sulphate, calcium hydrogen phosphate, magnesium oxide, calcium carbonate, tricalcium phosphate and magnesium carbonate.

46. A composition as claimed in any one of claims 17 to 45 having a pH in the range of about 3 to 7.

47. A composition as claimed in any one of claims 17 to 46 further comprising lactic acid, optionally in an amount of from 0.5 to 1.0 wt. %.

48. A composition as claimed in any one of claims 17 to 47 further comprising an acid buffer composition, optionally in an amount of from 0.5 to 0.8 wt. %.

49. A composition as claimed in any one of claims 17 to 48 further comprising at least one non-nutritive sweetener; optionally wherein the non-nutritive sweetener is present in an amount of up to about 0.1 wt. %, or up to about 0.05 wt. %, or up to about 0.025 wt. %.

50. A composition as claimed in any one of claims 17 to 49 further comprising:
i) one or more nutrient compositions, optionally one or more vitamins, minerals, fats or amino acids; and/or
ii) at least one acidulant, optionally at least one of malic acid and citric acid, further optionally in an amount of up to about 1.0 wt. %.

51. A composition as claimed in any one of claims 17 to 50 wherein the water content is at least 92 wt. %, at least 94 wt. % or at least 95 wt. %.

52. A composition as claimed in any one of claims 17 to 52 further comprising at least one flavourant, optionally in an amount of up to about 0.5 wt. %, or up to about 0.4 wt. %; and/or at least one colourant, optionally in an amount of up to about 0.5 wt. %, up to about 0.2 wt. %, or up to about 0.15 wt. %.

53. A gelled product prepared by gelation of a composition as claimed in any one of claims 17 to 52.
